(19) United States
(12) Patent Application Publication
(10) Pub. No.: US 2021/0145535 A1
(43) Pub. Date: May 20, 2021

(54) METHOD AND APPARATUS FOR BREATHING CIRCUIT ILLUMINATION

(71) Applicant: Torque Neutral Corporation, Margate, FL (US)

(72) Inventors: Boaz Barry Groman, Boca Raton, FL (US); Michael Black, Weston, FL (US)

(21) Appl. No.: 17/098,478

(22) Filed: Nov. 16, 2020

Related U.S. Application Data
(60) Provisional application No. 62/935,639, filed on Nov. 15, 2019.

(51) Int. Cl. A61B 90/35; A61M 16/01; F21K 2/06; F21V 21/088; A61M 16/08

(52) U.S. Cl. CPC A61B 90/35; A61M 16/01; A61M 2205/587; F21V 21/088; A61M 16/0883; F21K 2/06

(57) ABSTRACT

Method and apparatus for illuminating a patient's face during performing anesthesia (or other medical procedures) may comprise mounting a lighting device to a breathing circuit having a mask or an intubation tube. A clamp having two clips may be used to mount the lighting device, which may be a glowstick. One clip may hold the glow stick, while the other clip may mount the device to an appropriate portion of the breathing circuit. In use, the user (anesthesiologist) would activate the lighting device (e.g., glowstick) and attach (mount) the lighting device to the breathing circuit using the clamp.

### CROSS REFERENCE TO RELATED APPLICATIONS

Priority (filing date benefit) is claimed, from the following:
nonprovisional of 62/935,639 filed 15 Nov. 2019

### FIELD OF THE INVENTION

This disclosure relates to medical devices, more particularly to medical devices used during surgery, particularly during anesthesia, for providing breathing circuit illumination.

### BACKGROUND

A breathing circuit or breathing system is a medical device used to deliver oxygen, remove carbon dioxide, and deliver inhalational anesthetic agents to a patient. During a typical operation, the general/ceiling room lights are turned off and spotlights are utilized on the area-of-interest so that the surgeon(s) can focus. A drape is utilized to isolate the patient's head from the operation area, thereby providing the anesthesiologist with an isolated and splash-free place to administer the anesthesia. The anesthesia provider is therefore left in a darkened space. Although the anesthesia equipment provides lighted user interface, the light is dim and does not illuminate the patient's head region.

Currently anesthesia providers utilize a flashlight or cell phone to achieve the required illumination of a patient's face during laparoscopic or bariatric surgeries (actually all surgeries but these especially since the patient is operated on at an extreme incline—legs up & head down—which creates pressure to the head, so the face and eyes have to be intermittently examined). This requires one hand to be occupied, thereby limiting what an anesthesia provider can accomplish if manipulation of equipment becomes necessary; such as central lines, Swan Ganz Catheters, endotracheal tubes or NG tubes. Additionally, flashlights and cell phones are not sterilizable and introduce a contamination risk.

### SUMMARY

It is an object of the invention, generally, to provide better illumination during administering anesthesia, particularly in a hands-free manner.

According to the invention, generally, method and apparatus for illuminating a patient's face during performing anesthesia (or other medical procedures) may comprise mounting a lighting device to a breathing circuit having a mask or an intubation tube. A clamp having two clips may be used to mount the lighting device, which may be a glowstick. One clip may hold the glow stick, while the other clip may mount the device to an appropriate portion of the breathing circuit. In use, the user (anesthesiologist) would activate the lighting device (e.g., glowstick) and attach (mount) the lighting device to the breathing circuit using the clamp.

According to some embodiments (examples) of the invention, an apparatus for breathing circuit illumination may comprise: a clamp adapted to attach (for attaching) a lighting device to a breathing circuit. The clamp may comprise two clips, disposed back-to-back, one clip adapted for attaching the clamp to the breathing circuit, the other clip adapted for receiving (holding) the lighting device. The lighting device may be a glowstick.

Both clips may be C-shaped. One clip may be C-shaped, the other clip may be O-shaped. The clips may have a different size than one another. The two clips may be formed integrally with one another, back-to-back, and may be oriented with their axes approximately 90-degrees with respect to one another. The two clips may be formed integrally with one another, back-to-back, and an angular relationship between axes of the first and second portions may be fixed. The two clips may be formed separately from one another, and joined at their “bases” by an adhesive. The two clips may be formed separately from one another, and joined at their “bases” by mechanical means which allows the angular relationship between the two clips to be adjusted by a user.

According to some embodiments (examples) of the invention, a method for breathing circuit illumination may comprise: mounting a lighting device to a breathing circuit using the clamp of claim 1. The lighting device may be a glowstick. The clamp may comprise two clips which are adjustable with respect to one another.

According to some embodiments (examples) of the invention, an apparatus for illuminating a patient's face during performing anesthesia may comprise: a lighting device mounted to a breathing circuit by the clamp of claim 1.

Other objects, features and advantages of the invention may become apparent in light of the following description(s) thereof.

### BRIEF DESCRIPTION OF THE DRAWINGS

Reference will be made in detail to embodiments of the disclosure, non-limiting examples of which may be illustrated in the accompanying drawing figures (FIGs). The figures may generally be in the form of diagrams. Some elements in the figures may be stylized, simplified or exaggerated, others may be omitted, for illustrative clarity.

Although the invention is generally described in the context of various exemplary embodiments, it should be understood that it is not intended to limit the invention to these particular embodiments, and individual features of various embodiments may be combined with one another. Any text (legends, notes, reference numerals and the like) appearing on the drawings are incorporated by reference herein.

FIG. 1a is an illustration of a breathing circuit with a mask, incorporating the breathing circuit clamp of the invention.

FIG. 1b is an illustration of a breathing circuit with an intubation tube, incorporating the breathing circuit clamp of the invention.

FIG. 2 is an illustration of a breathing circuit with a mask, incorporating the breathing circuit clamp of the invention. Compare FIG. 1a.

FIGS. 3a, 3b are diagrams (perspective views, from different angles) of an embodiment of the breathing circuit clamp of the invention.

FIG. 4a is a diagram (perspective view) of an embodiment of the breathing circuit clamp of the invention.

FIG. 4b is a diagram showing the breathing circuit clamp of FIG. 4a attached (mounted) to a breathing circuit (“BC”) and holding a lighting device such as a glow stick (“GS”).

### DETAILED DESCRIPTION

Various embodiments (or examples) may be described to illustrate teachings of the invention(s), and should be construed as illustrative rather than limiting. It should be understood that it is not intended to limit the invention(s) to these particular embodiments. It should be understood that some individual features of various embodiments may be combined in different ways than shown, with one another. Reference herein to “one embodiment”, “an embodiment”, or similar formulations, may mean that a particular feature, structure, operation, or characteristic described in connection with the embodiment is included in at least one embodiment of the present invention. Some embodiments may not be explicitly designated as such (“an embodiment”).

The embodiments and aspects thereof may be described and illustrated in conjunction with systems, devices and methods which are meant to be exemplary and illustrative, not limiting in scope. Specific configurations and details may be set forth in order to provide an understanding of the invention(s). However, it should be apparent to one skilled in the art that the invention(s) may be practiced without some of the specific details being presented herein. Furthermore, some well-known steps or components may be described only generally, or even omitted, for the sake of illustrative clarity. Elements referred to in the singular (e.g., “a widget”) may be interpreted to include the possibility of plural instances of the element (e.g., “at least one widget”), unless explicitly otherwise stated (e.g., “one and only one widget”).

In the following descriptions, some specific details may be set forth in order to provide an understanding of the invention(s) disclosed herein. It should be apparent to those skilled in the art that these invention(s) may be practiced without these specific details. Any dimensions and materials or processes set forth herein should be considered to be approximate and exemplary, unless otherwise indicated. Headings (typically underlined) may be provided as an aid to the reader, and should not be construed as limiting.

The innovation disclosed herein is two pronged:
The first is the placement of a clamp (or bracket, or clip) onto the breathing circuit (mask, tubing, access ports, or any other breathing circuit feature) with the ability to hold an illumination (lighting) device.
The second component is the usage of a glowstick for illumination as a substitute for the flashlight or cell phone. The use of the glowstick makes the entire device single-use-disposable, thereby eliminating the expense of bulbs, wiring, batteries, chargers, etc., and increasing operational reliability. Of course, the clamp and glowstick can be integrated into a single component device and with any component of the breathing circuit.

With the use of a brightly illuminating glowstick, anesthesia providers (anesthetists) will have the ability to utilize a contamination-free solution to illuminate a patients face while maintaining use of both hands; further ensuring patient safety.

A breathing circuit generally includes a mask (which may be cone- or funnel-shaped), various tubing types, and access ports for various functions. The clamp described herein may snap-fit onto a tubular (cylindrical) portion of the mask or breathing circuit.

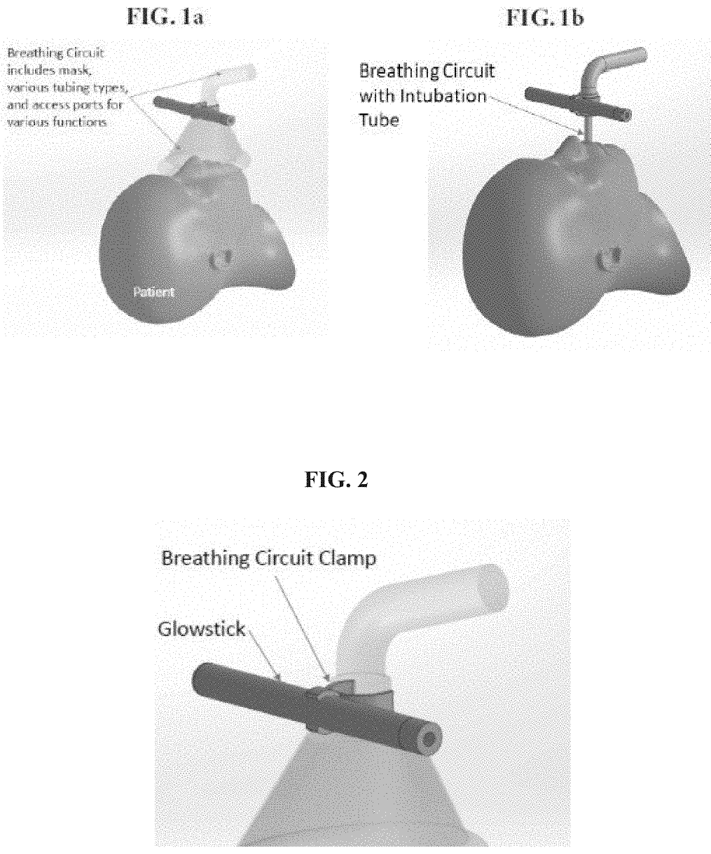

FIG. 1a shows a breathing circuit including a mask positioned on the face (mouth and nose area) of a patient being anesthetized. The breathing circuit may include the mask, various tubing types, and access ports for various functions.

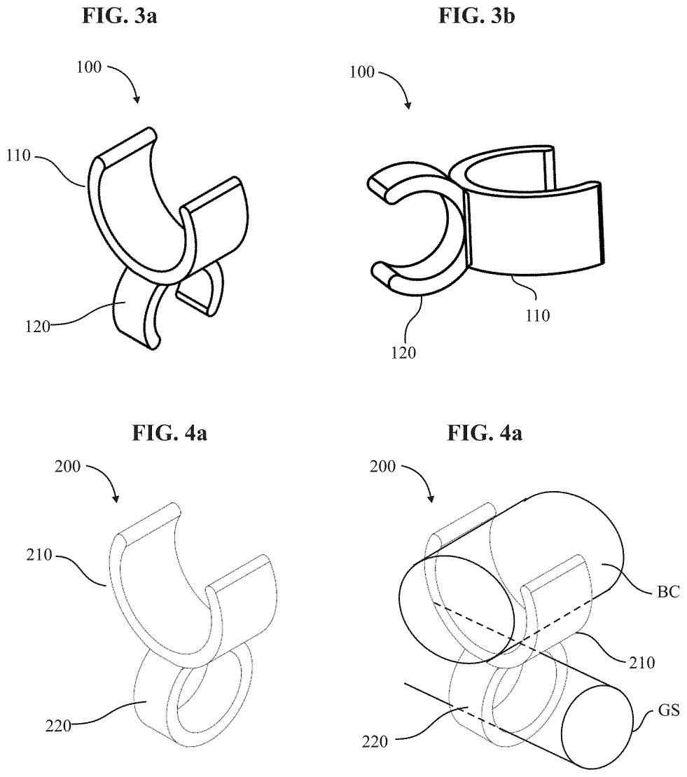

A glow stick (“GS”) is shown mounted with the clip (or clamp) of the present invention to an appropriate portion of the breathing circuit (“BC”), such as a cylindrical (rather than conical) portion of the mask. This is better viewed in FIG. 2. The clamp may be of the type shown in FIGS. 3a,b, or the type shown in FIGS. 4a,b.

FIG. 1b shows a breathing circuit (“BC”) with an intubation tube, rather than a mask. A glowstick (“GS”) is shown attached (mounted) to the breathing circuit intubation tube by a clamp, as may be described hereinbelow (e.g., in FIG. 4b).

The breathing circuit may include the mask and the intubation tube. A glow stick is shown mounted to an appropriate portion (such as the intubation tube) of the breathing circuit, in a manner similar to that shown in FIGS. 1a, 2.

FIG. 2 shows, in greater detail, the breathing circuit clamp releasably secured to an appropriate (such as cylindrical) portion of the breathing circuit, and holding a glowstick. (FIG. 1a version)

It may be noted that the glowstick is securely, yet releasably held in place by the clamp, and may be removed from the clamp for disposal or replacement.

FIGS. 3a,b show two views of the clamp 100 itself.

A portion (or clip) 120 is for securely holding a lighting device, such as a glow stick. A portion (or clip) 110 is for attaching (mounting) the glow stick to the breathing circuit.

The clamp 100 has two main portions, each of which may be referred to as a “clip”:
a first clip 110 for attaching (mounting) the clamp to the breathing circuit; and
a second clip 120 for holding a glowstick (or other illuminating device).

The clamp 100 may sometimes be referred to as a “clip device”, with the two portions 110, 120 may each be referred to as a “clip”. Hence, a “clip device” (or “clamp”) 100 may comprise two “clips” 110, 120.

Each of the clips 110, 120 may be formed like a “C”, such as a conventional cable or pipe clamp, and the two C-shaped portions (each having an axis) may be joined (or integrally formed) with one another, back-to-back, and oriented so that their axes are orthogonal to one another.

Generally, a C-shaped element, such as described herein, has two curved “arm” portions extending from a curved “base” (or “bight”) portion. This could also be considered to be a U-shaped element having curved base and arms portions.

Generally, the clip 110 has two arms that can deflect outwardly (apart fro one another) to engage a preferably tubular (cylindrical) portion of the breathing circuit.

Generally, the clip 120 has two arms that can deflect outwardly (apart fro one another) to receive a glowstick.

The clip 110 may be sized and shaped to fit (snap securely, yet releasably) onto a substantially cylindrical (tubular) portion of the breathing circuit. For example, the clip 110 may have a diameter of 0.84 inch (21.3 mm). The C-shaped clip 110 may be considered to be an incomplete circle, extending only 300° around, and having a 60° opening for mounting the clamp 100 to a tubular component of the breathing circuit.

The clip 120 may be sized and shaped to accept a substantially cylindrical light-emitting device, such as a glowstick or a flashlight. For example, the clip 120 may have a diameter of 0.56 inch (14.2 mm). The C-shaped clip 120 may be considered to be an incomplete circle, extending only 300° around, and having a 60° opening for inserting the glowstick into the clamp 100.

The inner diameter of the clip 110 may be 0.84 inch (21.3 mm). The inner diameter of the clip 120 may be 0.56 inch (14.2 mm). All dimensions set forth herein should be regarded as approximate, and exemplary. The clip 110 may be slightly larger (in diameter) than the clip 120. Other dimensions are possible, and the clip 110 may be smaller (in diameter) than the clip 120.

The dimensions of the clamp may be determined by the application—namely, the type of breathing apparatus to which the clamp is mounted, and the type of lighting device which is held by the clip.

In a “set” of clamp, different size clamps may be provided so that the user can adapt to different circumstances. The different size clamps may be color-coded, with different colors.

The clip 110 and the clip 120 of the clamp 100 may be formed integrally with one another. In this case, if it's all one piece, the angular relationship between the axes of the first and second portions would be fixed.

The clip 110 and the clip 120 of the clamp 100 may be formed separately from one another, and joined at their “bases” by an adhesive or by a rivet.

If the clip 110 and the clip 120 of the clamp 100 are joined with one another by mechanical means (such as a rivet), the angular relationship between the two clips could be adjusted by the user (i.e., the second portion could be rotated with respect to the first portion). For example, rather than the axes of the two clips being orthogonal to one another (as illustrated), their axes could be parallel with one another, or any angle therebetween.

The two clips 110, 120 may be joined together, back-to-back, with a “snap fit”, on clip having a projecting nub, the other clip having a recess for releasably receiving the nub. The two clips could be rotated with respect to one another, while remaining joined by the friction of the snap fit. Additionally, different size clips could be joined with one another to adapt to different size breathing circuit components and glow sticks (lighting devices), in which case the different size clips may conveniently be color-coded.

The orientations of the clip 110 and the clip 120 of the clamp 100 may be adjustable with respect to one another. The glow stick (“GS”) may be inserted into the appropriate clip (120, 220) of the overall clamp (100, 200) so that is sticks out evenly on both sides of the clamp, or in any other position that the glow stick may be securely held in place by the clamp, such as “biased” to one side or the other of the portion of the breathing circuit to which it is attached.

Generally, the clamp (100, 200) is intended to be very inexpensive to manufacture, and therefore disposable (single use).

The clamp (100, 200) may be made of a resilient (flexible) material, such as a plastic material, such as nylon, so that the “arms” of the C-shaped portions may deflect to be mounted upon the breathing circuit or to receive the glowstick.

The clamp (100, 200) may be autoclavable (sterilized), and therefore re-usable.

Although not shown, one or both of the clips (110/210, 120/220) of the clamp (100/200) may be adjustable (such as in diameter), and may be provided with means (such as a thumbscrew) to more securely mount the clamp (100/200) to the breathing circuit (“BC”) or to more securely grasp the glowstick (“GS”).

A thumbscrew could be incorporated into an “oversize” clip (110/210) so that it could be adapted to different size components of the breathing circuit (“BC”).

A thumbscrew could be incorporated into an “oversize” clip (120/220) so that a smaller diameter glow stick (“GS”) may securely be retained in the clip.

If a clip is slightly too big (in diameter) for the glow stick or the component of the breathing circuit, tape can be wrapped around the glow stick or the component of the breathing circuit so that the glow stick or the component of the breathing circuit fits better (more snugly) into the respective clip.

In use, the user (anesthesiologist) would typically activate the lighting device (e.g., glowstick) and attach (mount) the lighting device to the breathing circuit. This will illuminate the patient's face. In this regard, the term “breathing circuit illumination” may be taken to mean “illuminating a patient's face during performing anesthesia”.

FIGS. 4a,b show a variation of the clamp 200 itself. Generally the clamp 200 is the same as the clamp 100, but in this variation (embodiment), the clip 220 (compare 120) forms a complete circle (O-shaped), rather than being C-shaped. The clip 210 may be the same as the clip 110 in FIG. 3.

The clip 220 is for securely holding a lighting device, such as a glow stick. The clip 210 is for attaching (mounting) the glow stick to the breathing circuit.

FIG. 4b is a diagram showing the breathing circuit clamp 200 of FIG. 4a attached (mounted) to a breathing circuit (“BC”) and holding a lighting device such as a glow stick (“GS”).

While the invention(s) has/have been described with respect to a limited number of embodiments, these should not be construed as limitations on the scope of the invention(s), but rather as examples of some of the embodiments. Those skilled in the art may envision other possible variations, modifications, and implementations that are also within the scope of the invention(s), and claims, based on the disclosure(s) set forth herein.

What is claimed is:

1. Apparatus for breathing circuit illumination comprising:
a clamp adapted to attach a lighting device to a breathing circuit.

2. The apparatus of claim 1, wherein the clamp comprises:
two clips, disposed back-to-back, one clip adapted for attaching the clamp to the breathing circuit, the other clip adapted for holding the lighting device.

3. The apparatus of claim 2, wherein:
both clips are C-shaped.

4. The apparatus of claim 2, wherein:
one clip is C-shaped, the other clip is O-shaped.

5. The apparatus of claim 2, wherein:
the clips are different sizes than one another.

6. The apparatus of claim 2, wherein:
the two clips are formed integrally with one another, back-to-back, and are oriented with their axes approximately 90-degrees with respect to one another.

7. The apparatus of claim 2, wherein:
the two clips are formed integrally with one another, back-to-back, and an angular relationship between axes of the first and second portions is fixed.

8. The apparatus of claim 2, wherein:
the two clips are formed separately from one another, and joined at their “bases” by an adhesive.

9. The apparatus of claim 2, wherein:
the two clips are formed separately from one another, and joined at their “bases” by mechanical means which allows the angular relationship between the two clips to be adjusted by a user.

10. The apparatus of claim 1, wherein:
the lighting device is a glowstick.

11. Method for breathing circuit illumination comprising:
mounting a lighting device to a breathing circuit using the clamp of claim 1.

12. The method of claim 11, wherein:
the lighting device is a glowstick.

13. The method of claim 11, wherein:
the clamp comprises two clips which are adjustable with respect to one another.

14. Apparatus for illuminating a patient's face during performing anesthesia, comprising:
a lighting device mounted to a breathing circuit by the clamp of claim 1.
